(19) United States
(12) Patent Application Publication
(10) Pub. No.: US 2005/0221495 A1
(43) Pub. Date: Oct. 6, 2005

(54) Method and composition for improving NMR analysis of formation fluids

(76) Inventors: Stephen A. Bell, Houston, TX (US); William W. Shumway, Houston, TX (US); Kenneth W. Pober, Houston, TX (US); Peter Masak, West Chester, PA (US)

Correspondence Address: Karen B. Tripp;Attorney at Law, P.O. Box 1301, Houston, TX, 77251-1301, US

(21) Appl. No.: 10/813,719

(22) Filed: Mar. 31, 2004

(51) Int. Cl. G01N 33/03

(52) U.S. Cl. 436/60; 436/173

(57) ABSTRACT

A method and composition for distinguishing native hydrocarbons in a formation from oil-based drilling fluid with nuclear magnetic resonance (NMR) is provided so that NMR may be used in analyzing formation fluids from boreholes drilled with oil-based drilling fluids. In the method, the drilling fluid is doped with oil solubilized paramagnetic species before or during drilling of the borehole. The paramagnetic species cause a shift in the T1 and T2 NMR response of the drilling fluid.

### BACKGROUND OF THE INVENTION

1. Field of the Invention

The present invention relates to nuclear magnetic resonance analysis of subterranean formation fluids. Particularly, the present invention relates to distinguishing formation fluids from drilling fluids, particularly native oil from oil-based drilling fluids using a nuclear magnetic resonance tool. More particularly, the present invention relates to detecting oil mud contamination in formation fluids during analysis of the composition of said formation fluids.

2. Brief Description of Relevant Art

Nuclear magnetic resonance tools are known to be useful in measuring and assessing the composition of subterranean formation fluids. In nuclear magnetic resonance, the spins of atomic nuclei align themselves with an externally applied static magnetic field. This equilibrium situation can be disturbed by a pulse of an oscillating magnetic field (e.g., an RF pulse), which tips the spins away from the static field direction. After tipping, the spins simultaneously precess around the static field at the Larmor frequency and return to the equilibrium direction according to a decay time T1, which is the spin lattice relaxation time. Also associated with the spin of molecular nuclei is a second relaxation, T2, which is the spin-spin relaxation time. At the end of a ninety degree tipping (RF) pulse, all the spins are pointed in a common direction perpendicular to the static field, and they all precess near the Larmor frequency. However, because of molecular interactions, each nuclear spin precesses at a slightly different rate. T2 is a time constant of this dephasing.

Both brine or water and hydrocarbons in a subterranean formation produce NMR signals that may be detected in well logging with NMR tools. Ideally, the signals from water and hydrocarbons are separable so that the regions in the formation containing hydrocarbons can be identified. However, it is not always easy to distinguish the water signals from the hydrocarbon signals. Various methods have been used to separately identify water and hydrocarbon signals.

Currently, there are two general NMR methods of hydrocarbon detection that are commonly used. The simplest method involves making a small number of measurements (typically two) with one change in the measurement parameters. The difference between the measurements is then interpreted on the basis of known or assumed models for the NMR response of different fluids. The most common techniques of this type take advantage of the fact that water and hydrocarbons often have different relaxation times (T1 and/or T2) and diffusion constants.

The second category of NMR hydrocarbon detection methods is more general and applies forward modeling to NMR data acquired with different parameters, typically echo spacing and polarization time, although in principle the gradient may also be included as a parameter.

During well drilling, a drilling fluid or mud is pumped into the well and circulated to facilitate the drilling operation. The various functions of a drilling fluid include removing drill cuttings from the wellbore, cooling and lubricating the drill bit, aiding in support of the drill pipe and drill bit, and providing a hydrostatic head to maintain the integrity of the wellbore walls and prevent well blowouts. The drilling fluid may be water based or oil (or synthetic) based, with the specific drilling fluid system selected to optimize the drilling operation in accordance with the characteristics of the particular geological formation. As used herein, the term “oil-based” with respect to drilling fluids shall be understood to include synthetic oil or synthetic fluid based drilling fluids as well as drilling fluids having natural oils such as for example mineral oil or diesel oil as their base.

Filtration of such drilling fluids into the formation is a nuisance for NMR well logging operations. To prevent such filtration from distorting the results, core samples of the formation for laboratory testing must be taken at extended distances from the borehole in hopes that the drilling fluid filtrate has not reached that region of investigation and/or logging techniques must be used for differentiating the signals of the drilling fluid from those of the formation fluids. Several such techniques have been successfully practiced in wells drilled with water based drilling fluids. However, the contrast in the NMR tool response between oil-based drilling fluids and the formation oil can be very small, even below the noise level of the tool. Consequently, in wells drilled with oil-based fluids, drilling fluid filtration makes detection of hydrocarbons and estimation of residual oil saturation in the formation especially difficult or even impossible with NMR tools.

Nevertheless, because of the otherwise potential usefulness of NMR in analyzing formation fluids, the oil and gas industry continues to have an interest in new methods employing NMR tools and compensating for filtration of oil-based drilling fluid into the formation.

### SUMMARY OF THE INVENTION

The present invention provides a nuclear magnetic resonance (NMR) method for detecting the presence and preferably also the amount of any invasion or filtration of oil-based drilling fluid into a subterranean formation from a borehole penetrating the formation and drilled with the drilling fluid. That is, the present invention provides a method for distinguishing native or residual hydrocarbons in a formation from oil-based drilling fluid so that the drilling fluid does not distort the detection or measurement of such hydrocarbons using nuclear magnetic resonance.

In the method, oil solubilized “paramagnetic species” are added to the drilling fluid during drilling of the borehole. As used herein, the term “paramagnetic species” shall be understood to mean any chemical entity, molecule or ion comprising any transition metal, and/or lanthanium and/or other rare earth metal, that has paramagnetic character, and/or any persistent organic radical that has paramagnetic character. Persistent organic radicals are free electrons that “persist” or linger as free or do not quickly or immediately pair with other electrons. The paramagnetic species used in the invention are preferably selected from the group consisting of Fe³⁺, Mn²⁺, Ni²⁺, and Cu²⁺, Gd³⁺, and 2,2,6,6,-tetramethylpiperidineyl-1-oxyl (also called “TEMPO”) ions, and mixtures thereof, and are preferably selected with characteristics of the formation in mind so that the paramagnetic species selected will be of the type that will not interact with the formation. Preferably a sufficient amount of the paramagnetic species is used so that a quantitative determination of any drilling fluid that filters into the formation may be made as well as detection of the drilling fluid in the formation.

The paramagnetic species shorten the NMR/T1 and T2 responses of the oil or synthetic base comprising the drilling fluid to 10-100 milliseconds at typical formation or laboratory temperatures thereby causing the response to be sufficiently different from the NMR/T1 and T2 responses of native or residual hydrocarbons to distinguish them. Generally, the change in T1 and T2 relaxation rates for the drilling fluid is proportional to the concentration of paramagnetic species present.

The paramagnetic species may be added to the drilling fluid at any point during drilling before core sampling of the formation for laboratory testing or before running of a wireline NMR tool for downhole testing. Preferably, the paramagnetic species will be added to the drilling fluid at least about 200 feet before the point of the formation for testing is drilled.

The present invention also provides an oil-based drilling fluid that is readily distinguishable from native oil with NMR and methods for making such drilling fluid and for using such fluid in drilling for hydrocarbons.

### BRIEF DESCRIPTION OF THE DRAWINGS

FIG. 1 is a graph of the measure of the T2 (NMR) response as a function of the concentration of paramagnetic iron and manganese ions in an oil-based drilling fluid.

FIG. 2(a) is a graph of NMR data for an oil based drilling fluid.

FIG. 2(b) is a graph of the T2 inversion response of an oil-based drilling fluid.

FIG. 3(a) is a graph of NMR data for an oil-based drilling fluid doped with 384 ppm Fe³⁺ ions solubilized in an organic fluid.

FIG. 3(b) is a graph of a T2 inversion response for an oil-based drilling fluid doped with 384 ppm Fe³⁺ ions solubilized in an organic fluid.

FIG. 4(a) is a graph of NMR data for an oil-based drilling fluid doped with 384 ppm Mn²⁺ ions solubilized in an organic fluid.

FIG. 4(b) is a graph of a T2 inversion response for an oil-based drilling fluid doped with 384 ppm Mn²⁺ ions solubilized in an organic fluid.

### DETAILED DESCRIPTION OF PREFERRED EMBODIMENTS

In the present invention, a method is provided for enhancing the contrast in the NMR tool response between oil-based drilling fluids and the formation oil so that hydrocarbons may be detected, oil or residual oil saturation determined, and/or contamination by drilling fluid in the formation, may be analyzed with NMR tools.

The method of the invention requires doping of the oil-based drilling fluid with oil soluble or oil solubilized paramagnetic species. Generally, the greater the concentration of such paramagnetic species in the fluid, the greater the enhancement in the contrast in NMR response of the drilling fluid when compared to the NMR response of formation hydrocarbons. However, addition of even a small amount of oil soluble or oil solubilized paramagnetic species to oil-based drilling fluid causes a shift in the NMR response that distinguishes the drilling fluid from formation hydrocarbons. Preferably an amount of the paramagnetic species is used so that a quantitative determination of any drilling fluid that filters into the formation may be made as well as detection of the drilling fluid in the formation.

The paramagnetic species, from transition metals, lanthanium and other rare-earth metals, and/or persistent organic radicals, are preferably selected from the group consisting of Fe³⁺, Mn²⁺, Ni²⁺, and Cu²⁺, Gd³⁺, TEMPO ions, and mixtures thereof. Sources of paramagnetic species that may be useful in the present invention include without limitation Fe(NO₃)₃, MnO₂, NiSO₄, and CuSO₄, lanthanide, and other salts of transition metals or rare-earth minerals. Although many paramagnetic species are not typically considered soluble in oil, the paramagnetic species may be solubilized according to known techniques. For example, United Kingdom Patent Specification No. 879,991, of Victor Charles Ernest Burnop, entitled, “Metal-Organic Salt-Amine Complexes and Their Use in Hydrocarbon Oil Compositions,” incorporated herein by reference, teaches a process for preparing hydrocarbon oil-soluble complex metal salts that might be used. Other techniques that accomplish the result of oil solubilizing paramagnetic species may alternatively be used. Additionally, organic entities containing persistent radicals can be designed or formulated to be naturally soluble in oil-based fluids so that they do not have to be solubilized. TEMPO, for example, is naturally soluble in oil.

Preferably, the particular paramagnetic species selected for use is chosen with characteristics of the subterranean formation in mind so that the paramagnetic species selected will be of the type that will not interact with the formation. Manganese ions, for example, may interact with some formations.

The time for doping the oil-based drilling fluid with the paramagnetic species according to the invention will depend on the type of NMR testing planned and the purpose or end result of the testing. For example, if one or more cores are to be taken from the formation for laboratory NMR testing, perhaps for example to determine oil saturation, then the paramagnetic species should preferably be added to the drilling fluid before the borehole is drilled through the portion of the formation from which such cores are to be taken. For another example, if an NMR wireline tool is to be used for downhole testing in the borehole, then the paramagnetic species might be added to the drilling fluid before or at about the same time the drilling of the borehole is begun or at some later time during drilling of the borehole. Again, however, the paramagnetic species should be added to the drilling fluid before drilling through the portion of the formation to be tested.

Any oil-based drilling fluid may be used in the invention. In addition to, or instead of, adding paramagnetic species to the oil-based drilling fluid at some point during drilling, paramagnetic species may be added to the drilling fluid during formulation or preparation of the drilling fluid. The paramagnetic species may be added at any time during such preparation. Preferably, the particular paramagnetic species selected will be selected with the other components of the fluid in mind so that the paramagnetic species will not interact with any such components in such a way as to unfavorably alter the Theological characteristics of the fluid or any other characteristics of the fluid considered desirable for the intended use of the fluid. Further, the particular paramagnetic species selected will preferably be selected with the other components of the fluid in mind so that the paramagnetic species will not interact with any such components in such a way as to alter or interfere with the paramagnetic character of the paramagnetic species or the solubility or solubilization of the paramagnetic species in oil. Also, as previously noted, preferably the paramagnetic species chosen will be the type that will not interact with the formation in which the drilling fluid will be used.

A preferred method of drilling a borehole in a subterranean formation either containing hydrocarbons or in search for hydrocarbons, and in which use of an oil-based drilling fluid is desired, will use an oil-based drilling fluid comprising paramagnetic species soluble or solubilized in the oil base. Such method allows for logging of the formation and/or core sampling of the formation for analysis of the presence and/or quantity of hydrocarbons using nuclear magnetic resonance techniques at any time and at any point along the borehole.

Experiments were conducted that demonstrate or exemplify the invention.

Experimental

Commercially available forms of solubilized paramagnetic ions (Fe+++), CONOSTAN® Fe metallo-organo standard (doping agent) available from Conoco-Phillips, Inc. in Houston, Tex. and Ponca City, Okla., and solubilized paramagnetic manganese ions (Mn++), CONOSTAN® Mn metallo-organo standard available (doping agent) from Conoco-Phillips, Inc. in Houston, Tex. and Ponca City, Okla., were added to a commercially available synthetic drilling fluid base, ACCOLADE® synthetic oil base fluid available from Halliburton Energy Services, Inc. in Houston, Tex., at concentrations of 1000 ppm. The organo-metallic standards mixed readily with the drilling fluid base. The samples were allowed to stand for 18 hours at room temperature of 75° F. No (physical) separation occurred. The samples were then centrifuged at 10,000 RPM for 30 minutes and still no (physical) separation occurred.

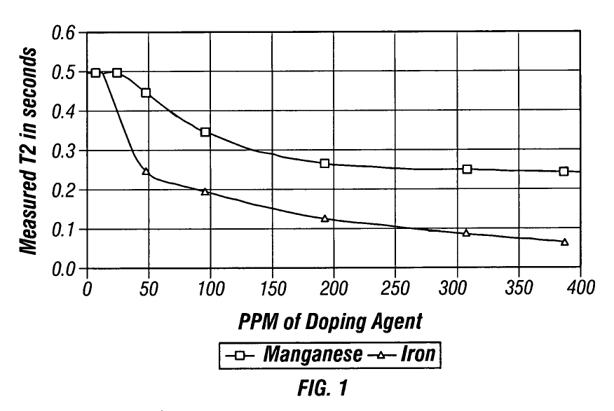

Samples of ACCOLADE® synthetic oil base containing (or doped with) different concentrations of CONOSTAN® Mn organo-metallic standard and samples of ACCOLADE® synthetic oil base containing different concentrations of CONOSTAN® Fe organo-metallic standard were prepared. These samples were tested with NMR equipment and the results are graphed in FIG. 1. The NMR relaxation times were significantly affected by the presence of the paramagnetic ion doping agent, even in relatively small concentrations. For example, just 50 ppm of the Mn++ reduced the T2 for the ACCOLADE® fluid about 50%.

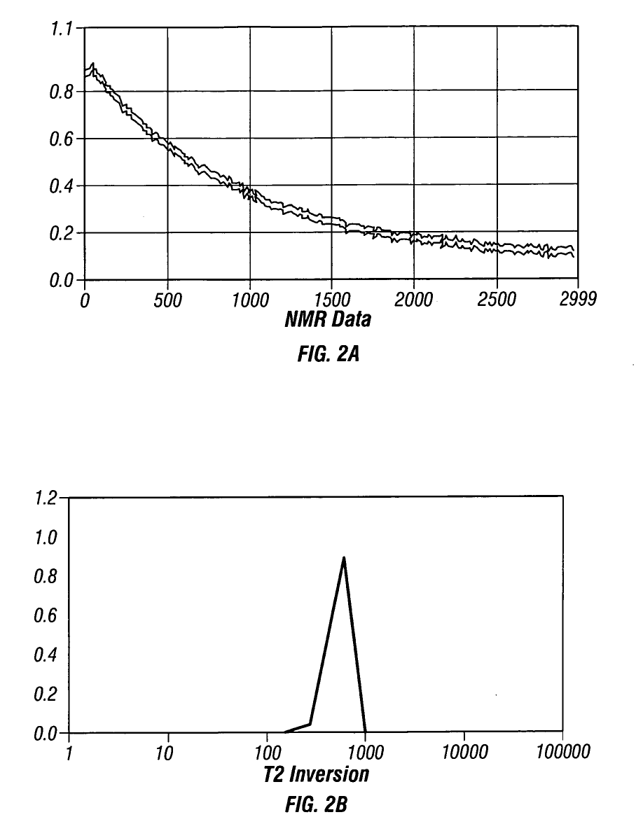

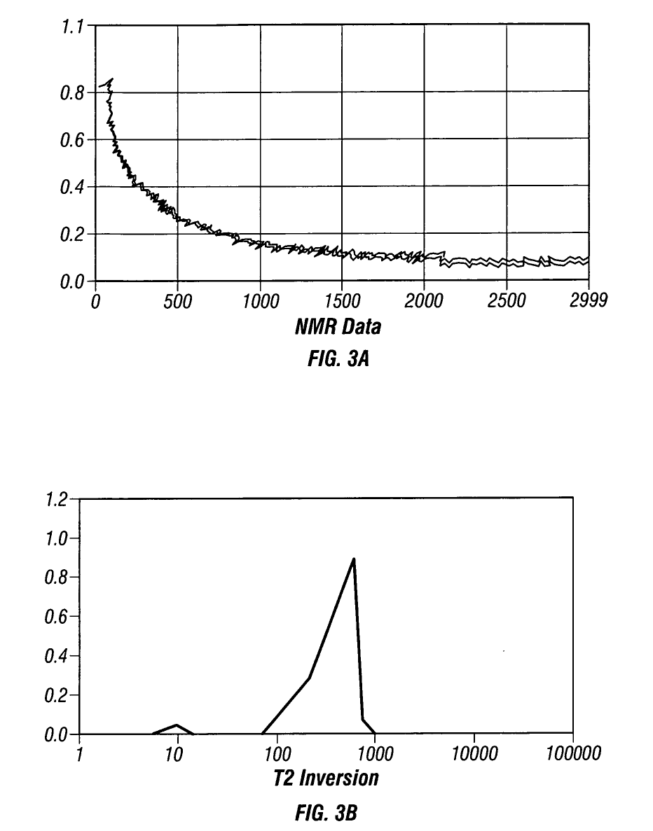

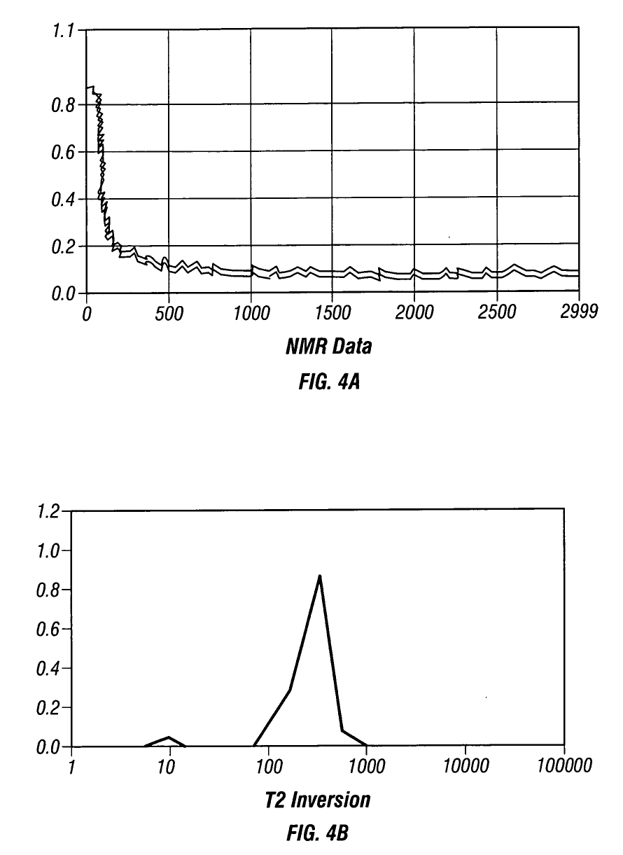

FIGS. 2, 3, and 4 compare the NMR data and T2 inversion for the ACCOLADE® drilling fluid without any dopant (FIG. 2(a) and FIG. 2(b)), and for the ACCOLADE® drilling fluid with 384 ppm iron ions (from CONOSTAN® Fe organo-metallic standard) added therein (FIG. 3(a) & FIG. 3(b)), and for the ACCOLADE® drilling fluid with 384 ppm manganese ions (from CONOSTAN® Mn organo-metallic standard) added therein (FIG. 4(a) and FIG. 4(b)). The effect of the dopant on the response of the drilling fluid is significant, demonstrating by this comparison the utility of adding the paramagnetic species to the drilling fluid as a tracer for detecting the drilling fluid in the formation.

The principles of the invention may be applied to techniques for taking NMR measurements with wireline tools run downhole in a borehole drilled with oil-based fluid, including without limitation logging while drilling tools. The principles of the invention may also be applied to laboratory techniques for NMR analysis of core samples taken from a subterranean formation. The principles of the invention may also be applied to techniques for taking NMR measurements downhole at locations of a formation where core samples are taken for further analysis or testing in the laboratory.

The foregoing description of the invention is intended to be a description of preferred embodiments. Various changes in the details of the described fluids and methods of preparation and use can be made without departing from the intended scope of this invention as defined by the appended claims.

1. A method of distinguishing oil based drilling fluid from subterranean formation fluid hydrocarbons during nuclear magnetic resonance testing, said method comprising adding paramagnetic species to the drilling fluid prior to said testing.

2. The method of claim 1 wherein said paramagnetic species comprises transition metals, rare earth metals, persistent organic radicals, or combinations thereof, having paramagnetic character.

3. The method of claim 1 wherein said paramagnetic species comprises Fe³⁺, Mn²⁺, Ni²⁺, and Cu²⁺, Gd³⁺, tetramethylpiperdinenyl-1-oxyl ions or combinations thereof.

4. The method of claim 1 wherein said paramagnetic species are oil solubilized.

5. The method of claim 1 wherein said paramagnetic species are oil soluble.

6. The method of claim 1 wherein said testing is in a borehole and said drilling fluid is circulated in the borehole after addition of said paramagnetic species and prior to said testing.

7. The method of claim 1 wherein said nuclear magnetic resonance measurements are made in an earth formation during logging while drilling operations.

8. A method of detecting hydrocarbon-bearing zones in a formation penetrated by a borehole drilled with oil-based drilling fluid, said method comprising adding paramagnetic species to said drilling fluid prior to or during the drilling of said borehole, circulating said fluid in said borehole; and acquiring nuclear magnetic resonance measurements of at least a portion of the formation.

9. The method of claim 8 wherein said nuclear magnetic resonance measurements are taken during logging while drilling operations.

10. The method of claim 8 further comprising taking at least one core sample from the region of said formation at which the nuclear magnetic resonance measurements were taken.

11. A method of detecting or identifying characteristics of hydrocarbons in the formation surrounding a borehole drilled with oil-based drilling fluid, said method comprising adding paramagnetic species to said drilling fluid prior to or during the drilling of said borehole, circulating said fluid in said borehole; and acquiring nuclear magnetic resonance measurements of at least a portion of the formation.

12. The method of claim 11 wherein said nuclear magnetic resonance measurements are taken during logging while drilling operations.

13. The method of claim 11 further comprising taking at least one core sample from the region of said formation at which the nuclear magnetic resonance measurements were taken.

14. A process of analyzing the fluid composition of a subterranean formation near a borehole drilled with oil-based fluid, said process comprising adding oil soluble or oil solubilized paramagnetic species to said oil-based fluid prior to or during said drilling of the borehole and detecting any filtrate of said drilling fluid in said formation using nuclear magnetic resonance.

15. The process of claim 14 wherein said paramagnetic species comprises transition metals, rare earth metals, persistent organic radicals, or combinations thereof, having paramagnetic character.

16. The process of claim 14 wherein said paramagnetic species comprises Fe³⁺, Mn²⁺, Ni²⁺ and Cu²⁺, Gd³⁺, tetramethylpiperdinenyl-1-oxyl ions, or combinations thereof.

17. The process of claim 14 wherein said analysis is conducted in the laboratory on cores of the formation sampled after said drilling with fluid to which said paramagnetic species were added.

18. The process of claim 14 wherein said analysis is conducted using measurements taken with a nuclear magnetic resonance tool in said borehole after said drilling with fluid to which said paramagnetic species were added.

19. A nuclear magnetic resonance process of analyzing the fluid composition of a subterranean formation near a borehole drilled with an oil-based drilling fluid containing paramagnetic species and detecting any filtrate of said drilling fluid in said formation using said paramagnetic species.

20. The process of claim 19 wherein said paramagnetic species comprises transition metals, rare earth metals, persistent organic radicals, or combinations thereof, having paramagnetic character.

21. The process of claim 19 wherein said paramagnetic species comprises Fe³⁺, Mn²⁺, Ni²⁺, and Cu²⁺, Gd³⁺, tetramethylpiperdinenyl-1-oxyl ions, or combinations thereof.

22. A drilling fluid comprising an oil base and paramagnetic species soluble in said base.

23. The drilling fluid of claim 22 wherein said oil base is a synthetic oil.

24. The drilling fluid of claim 22 wherein said oil base is a natural oil.

25. The drilling fluid of claim 22 wherein said paramagnetic species comprises transition metals, rare earth metals, persistent organic radicals, or combinations thereof, having paramagnetic character.

26. The drilling fluid of claim 22 wherein said paramagnetic species comprises Fe³⁺, Mn²⁺, Ni²⁺, and Cu²⁺, Gd³⁺, tetramethylpiperdinenyl-1-oxyl ions, or combinations thereof.

27. The drilling fluid of claim 22 wherein said paramagnetic species has been formulated to be soluble in oil.

28. The drilling fluid of claim 22 wherein said paramagnetic species has been oil solubilized.

29. A method of drilling for hydrocarbons, said method comprising using the drilling fluid of claim 22.

30. The method of claim 29 further comprising determining the location of said hydrocarbons using nuclear magnetic resonance.

31. The method of claim 29 further comprising analyzing the quantity of said hydrocarbons using nuclear magnetic resonance.

32. A method of drilling a borehole in a subterranean formation, said method comprising using an oil-based drilling fluid comprising paramagnetic species comprising transition metals, rare earth metals, persistent organic radicals, or combinations thereof, having paramagnetic character.

33. The method of claim 32 wherein said paramagnetic species in said drilling fluid are oil soluble.

34. The method of claim 32 wherein said paramagnetic species in said drilling fluid are oil solubilized.

35. The method of claim 32 further comprising logging said formation using a wireline nuclear magnetic resonance tool in said borehole.

36. The method of claim 35 further comprising using the logs from said logging to analyze the formation fluids.

37. The method of claim 32 further comprising taking core samples from said formation.

38. The method of claim 37 further comprising testing said core samples using nuclear magnetic resonance to analyze the formation fluids.

39. A method of preparing an oil-based drilling fluid, said method comprising providing an oil base and paramagnetic species soluble in said base.

40. The method of claim 39 wherein said oil base is a synthetic oil.

41. The method of claim 39 wherein said oil base is a natural oil.

42. The method of claim 39 wherein said paramagnetic species comprises transition metals, rare earth metals, persistent organic radicals, or combinations thereof, having paramagnetic character.

43. The method of claim 39 wherein said paramagnetic species comprises Fe³⁺, Mn²⁺, Ni²⁺, and Cu²⁺, Gd³⁺, tetramethylpiperdinenyl-1-oxyl ions, or combinations thereof.

44. The method of claim 39 wherein said paramagnetic species has been formulated to be soluble in oil.

45. The method of claim 39 wherein said paramagnetic species has been oil solubilized.
